(19) United States
(12) Patent Application Publication
(10) Pub. No.: US 2009/0057608 A1
(43) Pub. Date: Mar. 5, 2009

(54) ALKOXYLATE COMPOSITION AND A PROCESS FOR PREPARING THE SAME

(76) Inventors: Michiel Barend ELEVELD, Amsterdam (NL); Harmen Van Der Hulst, Amsterdam (NL); Jan Hermen Hendrik Meurs, Amsterdam (NL); Jasper Roelf Smit, Amsterdam (NL); Arie Van Zon, Amsterdam (NL)

Correspondence Address: Donald F. Haas;c/o Shell Oil Company, Intellectual Property, P.O. Box 2463, Houston, TX, 77252-2463, US

(21) Appl. No.: 12/146,231

(22) Filed: Jun. 25, 2008

(30) Foreign Application Priority Data
Jun. 27, 2007 (EP) 0711119735

(51) Int. Cl. C07C 29/10; C09K 3/00

(52) U.S. Cl. 252/182.12; 568/867

(57) ABSTRACT

A process for the preparation of an alkoxylate composition, said process comprising the steps of: (a) introducing into a reactor system one or more compounds with one or more active hydrogen atoms, selected from the group comprising alkanoic acids, alkanoic amides, alkanoic ethanolamides, alcohols and alkylmercaptans, and a double metal cyanide catalyst; (b) contacting the one or more compounds with one or more active hydrogen atoms and the double metal cyanide catalyst with propylene oxide and/or butylene oxide to form a first product mixture comprising double metal cyanide catalyst and compounds formed by the addition of one of more propylene oxide and/or butylene oxide units to the one or more compounds with one or more active hydrogen atoms; and (c) contacting the first product mixture with ethylene oxide to form a second product mixture comprising compounds formed by the addition of one of more ethylene oxide units to the compounds formed in step (b).

### FIELD OF THE INVENTION

The present invention relates to an alkoxylate composition and a process for preparing the same.

### BACKGROUND OF THE INVENTION

A large variety of products useful, for instance, as nonionic surfactants, wetting and emulsifying agents, solvents, in enhanced oil recovery (EOR) and as chemical intermediates, can be prepared by the addition reaction (alkoxylation reaction) of alkylene oxides (epoxides) with organic compounds having one or more active hydrogen atoms.

Such compounds are commonly made through an anionic alkylene oxide ring-opening process, whereby an alkylene oxide is combined with the compound having one or more active hydrogen atoms and a strongly basic catalyst such as potassium hydroxide or certain organic amines.

There are some disadvantages of using these strongly basic catalysts. One problem is that the catalysts produce a broader range of alkoxylated products than desirable for many applications. In addition, the basic catalyst often needs to be removed from the product before it is used, increasing manufacturing costs. Further, a strongly basic catalyst is incompatible with any compound having one or more active hydrogen atoms which also contains base-sensitive functional groups.

In order to overcome the problem of base-sensitive starting materials, Lewis acids such as boron trifluoride-diethyl etherate and organic amines such as triethylamine have been trialed as alkoxylation catalysts. Unfortunately, the use of these catalysts can lead to the formation of large amounts of by-products, especially when it is attempted to add three or more moles of alkylene oxide to the compound having one or more active hydrogen atoms. Such Lewis acid catalysts have a tendency to catalyze reactions wherein the growing polymer chain reacts with itself to form cyclic ethers. These by-products are difficult to remove from the desired product, preventing their use in many applications.

Double metal cyanide (DMC) catalysts have also been used for alkoxylation reactions. These catalysts help avoid problems caused by the rearrangement of propylene oxide which can occur in the presence of strongly basic catalyst.

Polypropoxylated starting compounds which are then end-capped with ethylene oxide are important raw materials in detergent formation as further derivatisation of the primary alcohols formed during ethoxylation is more efficient than derivatisation of the corresponding secondary alcohols formed by propoxylation. The ethoxylation of a previously formed poly(propoxylated) compound, in the presence of a DMC catalyst, is reported in EP 1200506.

An efficient process for the propoxylation and subsequent ethoxylation of organic compounds having one or more active hydrogen atoms, to form a narrow range of alkoxylated products would be highly desirable. Further, it would also be desirable if such a process could be adapted to allow facile derivatisation of the ethoxylated products to form compounds suitable for use in enhanced oil recovery.

### SUMMARY OF THE INVENTION

According to the present invention there is provided a process for the preparation of an alkoxylate composition, said process comprising the steps of:

(a) introducing into a reactor system one or more compounds with one or more active hydrogen atoms, selected from the group consisting of alkanoic acids, alkanoic amides, alkanoic ethanolamides, alcohols and alkylmercaptans, and a double metal cyanide catalyst;

(b) contacting the one or more compounds with one or more active hydrogen atoms and the double metal cyanide catalyst with propylene oxide and/or butylene oxide to form a first product mixture comprising double metal cyanide catalyst and compounds formed by the addition of one of more propylene oxide and/or butylene oxide units to the one or more compounds with one or more active hydrogen atoms; and

(c) contacting the first product mixture with ethylene oxide to form a second product mixture comprising compounds formed by the addition of one of more ethylene oxide units to the compounds formed in step (b).

Also according to the present invention there is provided an alkoxylate composition which comprises an alcohol having been reacted with one or more molar equivalents of PO and then one or more molar equivalents of EO.

### DETAILED DESCRIPTION OF THE INVENTION

It has now surprisingly been found that compounds suitable for use in enhanced oil recovery can be produced in an efficient process by firstly introducing into a reactor system one or more compounds with one or more active hydrogen atoms, selected from the group comprising alkanoic acids, alkanoic amides, alkanoic ethanolamides, alcohols and alkylmercaptans, and a DMC catalyst; then contacting the one or more compounds with one or more active hydrogen atoms and the DMC catalyst with propylene oxide and/or butylene oxide to form a first product mixture comprising compounds formed by the addition of one of more propylene oxide and/or butylene oxide units to the one or more compounds with one or more active hydrogen atoms; and then, without destroying the catalyst present in the first product mixture, contacting said mixture with ethylene oxide to form a second product mixture comprising DMC catalyst and compounds formed by the addition of one of more ethylene oxide units to the compounds of the first product mixture.

In a preferred embodiment of the present invention, without destroying the DMC catalyst present in the second product mixture, said mixture is then contacted with a functionalised epoxide to form a third product mixture comprising compounds formed by the addition of one of more functionalised epoxide units to the compounds which make up the second product mixture.

It will be readily understood from the above description that the same DMC catalyst will be present in each of the alkoxylation steps. In embodiments of the present invention, further DMC catalyst may be added for the later alkoxylation steps, in addition to the DMC catalyst already present.

The process of the present invention provides a method suitable for the formation of a narrow range of alkoxylated compounds. Furthermore, derivatisation of such compounds to the required detergent compounds can be achieved in a facile manner.

The alkoxylation reactions of the present invention proceed according to general equation I.

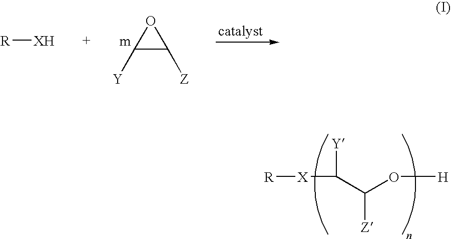

As used herein, R—XH represents a compound with one or more active hydrogen atoms. Y and Z correspond to the substituents on the epoxide. These may be H, methyl or ethyl in the case of ethylene oxide, propylene oxide and butylene oxide, or may be any substituent(s) on the functionalised epoxide. Substituents Y and Z will be present in the product compound as substituents Y′ and Z′. Y′ and Z′ may be identical to substituents Y and Z, respectively. However, it is possible that when reacting functionalised epoxides that some reaction or rearrangement of the original substituent(s) may occur.

It will be immediately apparent to one skilled in the art that the product of the reaction between a compound with one or more active hydrogen atoms and an epoxide results in an alkoxylated product which is itself a compound with one or more active hydrogen atoms and, therefore, both the starting material compound with one or more active hydrogen atoms and the alkoxylated product can react with any epoxide present in the reaction mixture. Thus, for any amount m of epoxide present added to the reaction mixture, a mixture of products with a range of values for n will be obtained. ‘A narrow range of alkoxylated compounds’ refers to the situation wherein for each step of the process of the present invention a narrow range of values for n is produced.

The process of the present invention may be carried out in any reactor system suitable for the alkoxylation of compounds with one or more active hydrogen atoms.

In general terms, suitable and preferred process temperatures and pressures for the purposes of this invention are the same as in conventional alkoxylation reactions between the same reactants, employing conventional catalysts. A temperature of at least about 90° C., particularly at least about 120° C. and most particularly at least about 130° C., may be utilized to achieve sufficient rate of reaction, while a temperature of about 250° C. or less, particularly about 210° C. or less, and most particularly about 190° C. or less, typically is desirable to minimize degradation of the product. As is known in the art, the process temperature can be optimized for given reactants, taking such factors into account.

Superatmospheric pressures, e.g., pressures between about 0.07 and about 1 MPa gauge (about 10 and about 150 psig), may be used.

The time required to complete this step of the process according to the invention is dependent both upon the degree of alkoxylation desired (i.e., upon the average alkylene oxide adduct number of the product) as well as upon the rate of the alkoxylation reaction (which is, in turn, dependent upon temperature, catalyst quantity and nature of the reactants). A typical reaction time may be from about 1 to about 24 hours for each step of the process.

The compound or compounds with one or more active hydrogen atoms may be selected from the group comprising alkanoic acids, alkanoic amides, alkanoic ethanolamides, alcohols and alkylmercaptans, or mixtures thereof.

Among the suitable alkanoic acids, particular mention may be made of the mono- and dicarboxylic acids, both aliphatic (saturated and unsaturated) and aromatic, and their carboxylic acid amide derivatives. Specific examples include lauric acid, myristic acid, palmitic acid, stearic acid, oleic acid, rosin acids, tall oil acids and terephthalic acid. Alkanoic amide derivatives of these compounds are also suitable.

Among the suitable alkylmercaptans, particular mention may be made of primary, secondary and tertiary alkane thiols having from 9 to 30 carbon atoms, particularly those having from 9 to 20 carbon atoms. Specific examples of suitable tertiary thiols are those having a highly branched carbon chain which are derived via hydrosulphurisation of the products of the oligomerisation of lower olefins, particularly the dimers, trimers, tetramers and pentamers of propylene and the butylenes. Secondary thiols are exemplified by the products of the hydrosulphurisation of the substantially linear oligomers of ethylene as are produced by the Shell Higher Olefins Process. Representative, but by no means limiting, examples of thiols derived from ethylene oligomers include the linear carbon chain products, such as 2-decanethiol, 3-decanethiol, 4-decanethiol, 5-decanethiol, 3-dodecanethiol, 4-decanethiol, 5-decanethiol, 3-dodecanethiol, 5-dodecanethiol, 2-hexadecanethiol, 5-hexadecanethiol, and 8-octadencanethiol, and the branched carbon chain products, such as 2-methyl-4-tridecanethiol. Primary thiols are typically prepared from terminal olefins by hydrosulphurisation under free-radical conditions and include, for example, 1-dodecanethiol, 1-tetradecanethiol and 2-methyl-1-tridecanethiol.

Aromatic alcohols, such as phenols, may also be suitable. Among the phenols, particular mention may be made of phenol and alkyl-substituted phenols wherein each alkyl substituent has from 3 to 30 (preferably from 3 to 20) carbon atoms, for example, p-hexylphenol, p-nonylphenol, p-decylphenol, nonylphenol and didecyl phenol.

In a preferred embodiment, the compound with one or more active hydrogen atoms is a hydroxyl-containing reactant. More preferably, the compound with one or more active hydrogen atoms is an alcohol or a mixture of alcohols.

Suitable starting alcohols for use in the process of the present invention include those known in the art for reaction with alkylene oxides and conversion to alkoxylated alcohol products, including both mono- and poly-hydroxy alcohols.

Acyclic aliphatic mono-hydric alcohols (alkanols) form a most preferred class of reactants, particularly the primary alkanols, although secondary and tertiary alkanols are also very suitably utilized in the preparation of the alkoxylated alcohol composition herein. As is often the case for alkoxylation reactions, primary alcohols are more reactive, and in some cases substantially more reactive, than the corresponding secondary and tertiary compounds. However, DMC catalysts may be used for the alkoxylation of secondary alcohols as well as primary alcohols.

Secondary alcohols suitable for use in the present invention can be derived from relatively cheap feedstocks such as paraffins (by oxidation). Suitable paraffins for producing secondary alcohols are, for example, those produced from Fischer-Tropsch technologies.

Preference can also be expressed, for reasons of both process performance and commercial value of the product, for alkanols having from 8 to 36 carbon atoms, with C₉ to C₂₄ alkanols considered more preferred and C₁₂ to C₂₄ alkanols and mixtures thereof being considered most preferred.

As a general rule, the alkanols may be of branched or straight chain structure depending on the intended use. In one embodiment, preference further exists for alkanol reactants in which greater than 50 percent, more preferably greater than 60 percent and most preferably greater than 70 percent of the molecules are of linear (straight chain) carbon structure. In another embodiment, preference further exists for alkanol reactants in which greater than 50 percent, more preferably greater than 60 percent and most preferably greater than 70 percent of the molecules are of branched carbon structure.

The general suitability of such alkanols as reactants in alkoxylation reactions is well recognized in the art. Commercially available mixtures of primary monohydric alkanols prepared via the oligomerisation of ethylene and the hydroformylation or oxidation and hydrolysis of the resulting higher olefins are particularly preferred. Examples of commercially available alkanol mixtures include the NEODOL® Alcohols, trademark of and sold by Shell Chemical Company, including mixtures of C₉, C₁₀ and C₁₁ alkanols (NEODOL® 91 Alcohol), mixtures of C₁₂ and C₁₃ alkanols (NEODOL® 23 Alcohol), mixtures of C₁₂, C₁₃, C₁₄ and C₁₅ alkanols (NEODOL® 25 Alcohol), mixtures of C₁₄ and C₁₅ alkanols (NEODOL® 45 Alcohol and NEODOL® 45E Alcohol), mixtures of C₁₆ to C₁₇ alkanols (NEODOL® 67 Alcohol) and mixtures of C₁₆ to C₁₉ alkanols; the ALFOL Alcohols (ex. Vista Chemical Company), including mixtures of C₁₀ and C₁₂ alkanols (ALFOL 1012 alkanol), mixtures of C₁₂ and C₁₄ alkanols (ALFOL 1214 alkanol), mixtures of C₁₆ and C₁₈ alkanols (ALFOL 1618 alkanol), and mixtures of C₁₆, C₁₈ and C₂₀ alkanols (ALFOL 1620 alkanol), the EPAL Alcohols (Ethyl Chemical Company), including mixtures of C₁₀ and C₁₂ alkanols (EPAL 1012 alkanol), mixtures of C₁₂ and C₁₄ alkanols (EPAL 1214 alkanol), and mixtures of C₁₄, C₁₆ and C₁₈ alkanols (EPAL 1418 alkanol), and the TERGITOL-L Alcohols (Union Carbide), including mixtures of C₁₂, C₁₃, C₁₄ and C₁₅ alkanols (TERGITOL-L 125 alkanol). Also suitable for use herein is NEODOL® 1 alcohol, which is primarily a C₁₁ alkanol. Also very suitable are the commercially available alkanols prepared by the reduction of naturally occurring fatty esters, for example, the CO and TA products of Proctor and Gamble Company and the TA alcohols of Ashland Oil Company.

As mentioned above, secondary alcohols are also a suitable class of reactants for use herein. Examples of secondary alcohols suitable for use herein include 2-undecanol, 2-hexanol, 3-hexanol, 2-heptanol, 3-heptanol, 2-octanol, 3-octanol, 2-nonanol, 2-decanol, 4-decanol, 2-dodecanol, 2-tetradecanol, 2-hexadecanol, and mixtures thereof.

Mixtures of alcohols comprising primary and secondary alcohols are also suitable for use herein.

In particular, oxidation products arising from Fischer-Tropsch derived paraffins (which may include mixtures of primary and secondary alcohols) are particularly suitable for use herein.

It has been found that a particularly suitable alkoxylated product comprises an alcohol which has been reacted with one or more molar equivalents of propylene oxide followed by one or more molar equivalents of ethylene oxide. Thus, one embodiment of the present invention is directed to such an alkoxylate composition. Preferably, the alcohol used in this embodiment of the present invention is a branched primary alcohol composition, having from 8 to 36 carbon atoms and an average number of branches per molecule of at least 0.7, said branching comprising methyl and ethyl branches. More preferably, the alcohol is a branched primary alcohol composition, having from 14 to 21 carbon atoms and an average number of branches per molecule of from 0.7 to 3.0, said branching comprising methyl and 5-30% ethyl branches and 5-25% branching at the carbon atom adjacent to the hydroxyl carbon atom, said composition comprising less than 0.5 atom % of quaternary carbon atoms.

After the compound with one or more active hydrogen atoms and the DMC are introduced into the reactor system. These compounds are contacted with propylene and/or butylene oxide in order to form a first product mixture comprising DMC catalyst and compounds formed by the addition of one or more propylene and/or butylene oxide units to the compound with one or more active hydrogen atoms, i.e. compounds of general formula (II), wherein R′ is methyl and/or ethyl.

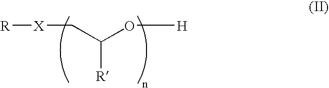

In a preferred embodiment of the process of the present invention, the propylene oxide and/or butylene oxide is contacted with the alcohol in a molar ratio in the range of from 2 to 20 moles of propylene oxide and/or butylene oxide per mole of alcohol. More preferably, the propylene oxide and/or butylene oxide is contacted with the alcohol in a molar ratio in the range of from 3 to 12 moles of propylene oxide and/or butylene oxide per mole of alcohol.

As explained above, the alkoxylation of a compound with one or more active hydrogen atoms leads to a mixture of products. It will be readily understood that such a mixture will contain compounds for which n may be a wide range of whole numbers, and also zero.

After formation of the first product mixture, said mixture is contacted with ethylene oxide in order to form a second product mixture comprising DMC catalyst and compounds (i.e. compounds of general formula (III), wherein R′ is methyl and/or ethyl) formed by the addition of one or more ethylene oxide units to the compounds present in the first product mixture

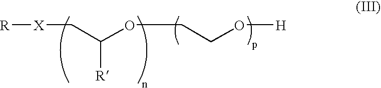

As explained above, the product mixture will comprise a mixture of compounds having a range of values for n and p.

Preferably, the ethylene oxide is contacted with the propoxylated and/or butoxylated alcohol in a molar ratio in the range of from 1 to 9 moles of ethylene oxide per mole of alcohol.

After formation of the second product mixture, in a preferred embodiment of the process of the present invention said second product mixture is contacted with a functionalised epoxide in order to form a third product mixture comprising compounds (i.e. compounds of general formula (IV), wherein R′ is methyl and/or ethyl) formed by the addition of one or more functionalised epoxide units to the compounds present in the second product mixture.

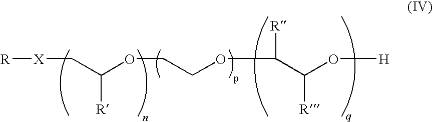

R″ and R′″ will be groups according to the substitution of the functionalised epoxide. As stated above R″ and R′″ may comprise the substituents of the functionalised epoxide as present in the functionalised epoxide itself, or they may comprise groups formed by reaction or rearrangement of such substituents under the conditions of the alkoxylation reaction. Further, as explained above, the product mixture will comprise a mixture of compounds having a range of values for n, p and q.

Preferably, the functionalised epoxide is contacted with the ethoxylated and propoxylated and/or butoxylated alcohol in a molar ratio in the range of from 1 to 4 moles of functionalised epoxide per mole of alcohol.

Suitably, the functionalised epoxide is selected from the group comprising epihalohydrins, glycidol derivatives, epoxidised acrylic or methacrylic acid derivatives and diene monoepoxides.

The catalyst used for the preparation of the alkoxylate composition of the present invention is a double metal cyanide catalyst. Any double metal cyanide catalyst suitable for use in alkoxylation reactions can be used in the present invention. Conventional DMC catalysts are prepared by reacting aqueous solutions of metal salts and metal cyanide salts or metal cyanide complex acids to form a precipitate of the DMC compound.

The DMC catalysts used herein are particularly suitable for the direct ethoxylation of secondary alcohols.

The catalyst may be used in an amount which is effective to catalyze the alkoxylation reaction. The catalyst may be used at a level such that the level of solid DMC catalyst remaining in the final alkoxylate composition is in the range from about 1 to about 1000 ppm (wt/wt), preferably of from about 5 to about 200 ppm (wt/wt), more preferably from about 10 to about 100 ppm (wt/wt). The DMC catalysts used in the present invention are very active and hence exhibit high alkoxylation rates. They are sufficiently active to allow their use at very low concentrations of the solid catalyst content in the final alkoxylation product composition. At such low concentrations, the catalyst can often be left in the alkoxylated alcohol composition without an adverse effect on product quality. The ability to leave catalysts in the alkoxylated alcohol composition is an important advantage because commercial alkoxylated alcohols currently require a catalyst removal step. The concentration of the residual cobalt in the final alkoxylate composition is preferably below about 10 ppm (wt/wt).

Suitable metal salts and metal cyanide salts are, for instance, described in U.S. Pat. No. 5,627,122 and U.S. Pat. No. 5,780,584 which are herein incorporated by reference in their entirety. Thus, suitable metal salts may be water-soluble salts suitably having the formula M(X′)ₙ′, in which M is selected from the group consisting of Zn(II), Fe(II), Ni(II), Mn(II), Co(II), Sn(II), Pb(II), Fe(III), Mo(IV), Mo(VI), Al(III), V(V), V(IV), Sr(II), W(IV), W(VI), Cu(II), and Cr(III). More preferably, M is selected from the group consisting of Zn(II), Fe(II), Co(II), and Ni(II), especially Zn(II). In the formula, X′ is preferably an anion selected from the group consisting of halide, hydroxide, sulfate, carbonate, cyanide, oxalate, thiocyanate, isocyanate, isothiocyanate, carboxylate, and nitrate. The value of n′ satisfies the valency state of M and typically is from 1 to 3. Examples of suitable metal salts include, but are not limited to, zinc chloride, zinc bromide, zinc acetate, zinc acetonylacetate, zinc benzoate, zinc nitrate, iron(II) chloride, iron(II) sulfate, iron(II) bromide, cobalt(II) chloride, cobalt(II) thiocyanate, nickel(II) formate, nickel(II) nitrate, and the like, and mixtures thereof. Zinc halides, and particularly zinc chloride, are preferred.

In one embodiment, the metal cyanide salt may be a water-soluble metal cyanide salt having the general formula (Y)ₐ′M′(CN)_{b}′(A′)_{c}′ in which M′ is selected from the group consisting of Fe(II), Fe(III), Co(II), Co(III), Cr(II), Cr(III), Mn(II), Mn(III), Ir(III), Ni(II), Rh(III), Ru(II), V(IV), and V(V). More preferably, M′ is selected from the group consisting of Co(II), Co(III), Fe(II), Fe(III), Cr(III), Ir(III), and Ni(II), especially Co(II) or Co(III). The water-soluble metal cyanide salt may contain one or more of these metals. In the formula, Y is an alkali metal ion or alkaline earth metal ion, such as lithium, sodium, potassium and calcium. A′ is an anion selected from the group consisting of halide, hydroxide, sulfate, carbonate, cyanide, oxalate, thiocyanate, isocyanate, isothiocyanate, carboxylate, and nitrate. Both a′ and b′ are integers greater than or equal to 1; c′ can be 0 or an integer; the sum of the charges of a′, b′, and c′ balances the charge of M′. Suitable water-soluble metal cyanide salts may include, for example, potassium hexacyanocobaltate(III), potassium hexacyanoferrate(II), potassium hexacyanoferrate(III), calcium hexacyanocobaltate(III) and lithium hexacyanoiridate(III). A particularly preferred water-soluble metal cyanide salt for use herein is potassium hexacyanocobaltate(III).

DMC catalysts useful in the process of this invention may be prepared according to the processes described in US 2005/0014979 which is herein incorporated by reference in its entirety.

DMC catalysts may be prepared in the presence of a low molecular weight organic complexing agent such that a dispersion is formed comprising a solid DMC complex in an aqueous medium. The organic complexing agent used should generally be reasonably to well soluble in water. Suitable complexing agents are, for instance, disclosed in U.S. Pat. No. 5,158,922, which is herein incorporated by reference in its entirety, and in general are water-soluble heteroatom-containing organic compounds that can complex with the double metal cyanide compound. Thus, suitable complexing agents may include alcohols, aldehydes, ketones, ethers, esters, amides, ureas, nitriles, sulfides, and mixtures thereof.

Combining both aqueous reactant streams may be conducted by conventional mixing techniques including mechanical stirring and ultrasonic mixing. Although applicable, it is not required that intimate mixing techniques like high shear stirring or homogenization are used. The reaction between metal salt and metal cyanide salt may be carried out at a pressure of from about 50 to about 1000 kPa and a temperature of from about 0 to about 80° C. However, it is preferred that the reaction be carried out at mild conditions, i.e. a pressure of about 50 to about 200 kPa and a temperature of from about 10 to about 40° C.

After the reaction has taken place and a DMC compound has been formed an extracting liquid may be added to the dispersion of solid DMC complex in aqueous medium, in order that the DMC catalyst particles may be efficiently and easily separated from the aqueous phase without losing any catalytic activity.

Suitable extracting liquids are described in U.S. Pat. No. 6,699,961 which is herein incorporated by reference in its entirety. A suitable extracting liquid should meet two requirements: firstly it should be essentially insoluble in water and secondly it must be capable of extracting the DMC complex from the aqueous phase. The extracting liquid can, for instance, be an ester, a ketone, an ether, a diester, an alcohol, a di-alcohol, a (di)alkyl carbamate, a nitrile or an alkane. An especially preferred extracting liquid for use herein is methyl tert-butyl ether.

Typically the extracting liquid is added under stirring and stirring is continued until the liquid has been uniformly distributed through the reaction mixture. After the stirring has stopped the reaction mixture is allowed sufficient time to settle, i.e. sufficient time to separate into two phases: an aqueous bottom layer and a layer floating thereon containing the DMC catalyst dispersed in the extracting liquid.

The next part of the catalyst preparation process is for the aqueous layer to be removed. Since the aqueous layer forms the bottom layer of the two phase system formed, this may be easily accomplished by draining the aqueous layer via a valve in the bottom part of the vessel in which the phase separation occurred. After removal of the aqueous phase, the remaining phase contains the solid DMC catalyst particles which are dispersed or finely divided in the extracting compound and which are subsequently recovered.

The catalyst recovery step may be carried out in various ways. The recovery procedure may involve mixing the DMC catalyst with complexing agent, optionally in admixture with water, and separating DMC catalyst and complexing agent/water again, e.g. by filtration, centrifugation/decantation or flashing. This procedure may be repeated one or more times. Eventually, the catalyst may be dried and recovered as a solid. The recovery step may comprise adding a water/complexing agent to the DMC catalyst layer and admixing catalyst layer and water/complexing agent (e.g. by stirring), allowing a two-phase system to be formed and removing the aqueous layer. This procedure may be repeated one to five times after which the remaining catalyst layer may be dried and the catalyst may be recovered in solid form (as a powder) or, alternatively, a liquid alcohol/polyol may be added to the catalyst layer and a catalyst suspension in liquid alcohol is formed, which may be used as such.

The alcohol/polyol added may be any liquid alcohol/polyol, which is suitable to serve as a liquid medium for the DMC catalyst particles. When the DMC catalyst is used for catalyzing the alkoxylation reaction of alcohols, it is preferred to use an alcohol/polyol which is compatible with the alkoxylated alcohols to be produced and which will not have any negative effect on the final alkoxylated alcohol produced when present therein in trace amounts. Examples of suitable polyols include polyols such as polyethylene glycol and polypropylene glycol.

The organic complexing agent may be removed from the catalyst slurry. This may be achieved by any means known in the art to be suitable for liquid-liquid separation. A preferred method for the purpose of the present invention is flashing off the complexing agent at atmospheric conditions or under reduced pressure. Flashing under reduced pressure is preferred, as this enables separation at a lower temperature, which reduces the risk of thermal decomposition of the DMC catalyst.

The DMC catalyst may be recovered as a slurry in liquid alcohol/polyol. The advantage of such a slurry is that it is storage stable and may, for instance, be stored in a drum. Moreover, dosing of the catalyst and its distribution through the alkoxylation medium is greatly facilitated by using a catalyst slurry.

The following non-limiting Examples will illustrate the invention.

### EXAMPLES

### Example 1

### Preparation of a 3% wt DMC Catalyst Slurry in NEODOL 67 Alcohol

9.00 g of solid DMC catalyst, prepared according to example 2 of EP 1663928, which is herein incorporated by reference in its entirety, is added to a beaker. Subsequently 291.3 g of NEODOL® 67 alcohol is added at room temperature.

The mixture is stirred for 5 minutes with a high speed high shear stirrer (Ultraturrax) to give a 3% wt DMC catalyst in NEODOL® 67 alcohol slurry.

### Example 2

### Preparation of a Propoxylated-Ethoxylated Branched C16-17 Alcohol Having an Average of 7 Propyleneoxy and 2 Ethyleneoxy Groups Per Molecule

A 1-litre stirred tank reactor was charged with 273.94 g of NEODOL® 67 alcohol and 0.545 g of the 3% wt the DMC catalyst slurry in NEODOL® 67 alcohol, formed by the process described in Example 1, to attain 20 ppm wt/wt solid DMC catalyst based on end product. Under constant stirring, the reactor tank was flushed three times with nitrogen, by raising the pressure within the reactor tank to 2 bara by addition of nitrogen and subsequently releasing the pressure to atmospheric pressure. The reactor contents were heated, under a nitrogen atmosphere, to a temperature of 130° C. and subsequently stripped by applying a vacuum and a nitrogen purge at a pressure of 100 mbara.

After 1 hour, the nitrogen purge and vacuum was stopped and 437.4 g of propylene oxide (PO) was added over a period of approximately 2.5 hours. The pressure which was 0.6 bara at begin of addition decreased to 0.2 bara and slowly increased to 0.42 bara over the course of the addition. After all the PO had been introduced into the reactor contents, the reactor contents were held at the reaction temperature for half an hour, allowing the reaction of residual PO. The pressure dropped to 0.31 bara.

Subsequently, the pressure was increased to 1.5 bara by adding nitrogen and 94.8 g of ethylene oxide (EO) was introduced over a period of approximately 33 minutes. During this addition the pressure increased to 2.0 bara.

After all of the EO had been introduced to the reactor contents, the reactor contents were held at the reaction temperature for a further 0.5 hour, allowing the reaction of residual EO and giving a pressure decrease to 1.75 bara. Subsequently, any residual EO was stripped off with nitrogen at 30 mbara for 15 minutes.

### Example 3

### Preparation of a Propoxylated-Ethoxylated (Linear) Cetyl/Stearyl Alcohol Having an Average of 7 Propyleneoxy and 2 Ethyleneoxy Groups Per Molecule

A 1-litre stirred tank reactor was charged with 278.8 g of cetyl/stearyl alcohol¹ preheated to 80° C. and 0.554 g of the 3% wt DMC catalyst slurry in NEODOL® 67 alcohol formed by the process of Example 1, to attain 20 ppm wt/wt solid DMC catalyst based on end product. Under constant stirring, the reactor tank was flushed three times with nitrogen, by raising the pressure within the reactor tank to 2 bara and subsequently releasing the pressure to atmospheric pressure. The reactor contents were heated, under a nitrogen atmosphere, to a temperature of 130° C. and subsequently stripped by applying a vacuum and a nitrogen purge at a pressure of 100 mbara. ¹ TA-1618F KU, batch FPG-6231-6102 from The Proctor & Gamble Distributing Company, Cincinnati, Ohio 45241 USA, having the following analysis according to its certificate of analysis: Hydroxyl Value=211; Acid Value=0.3; Saponification Value=0.3; Iodine Value=0.6; Color, APHA=5; Moisture, %=0.1; Melting Point, ° C.=53; Chain length Distribution by GC in wt %, C14OH&lower=0.2, C16OH=31, C18OH=67, C20&higher=0.0, Hydrocarbon=0.0

After 1 hour the nitrogen purge and vacuum was stopped and 428.6 g of PO was added in about 2.5 hours. The pressure which raised to 0.7 bara at begin of addition decreased to 0.2 bara and slowly increased to 0.48 bara over the course of the addition. After all the PO had been introduced into the reactor contents, the reactor contents were held at the reaction temperature for half an hour, allowing the reaction of residual PO. The pressure dropped to 0.34 bara. The pressure was increased to 1.5 bara by adding nitrogen and 92.9 g of EO was introduced in about 34 minutes. The pressure increased to 2.0 bara.

After all of the EO had been introduced to the reactor contents, the reactor contents were held at the reaction temperature for a further 0.5 hour, allowing the reaction of residual EO and giving a pressure decrease to 1.74 bara. Subsequently, any residual EO was stripped off with nitrogen at 30 mbara for 15 minutes.

TABLE 1
Analytical data

| | Example 2 | Example 3 |
|---|---|---|
| OH-value | 75.1 mg KOH/g | 73.8 mg KOH/g |
| Unsaturation | <10 mmol/kg | <10 mmol/kg |
| Viscosity (40° C.) | 41.4 cSt | 39.0 cSt |
| Water content | 0.01% | 0.01% |
| Acid content | 0.06 mg KOH/g | 0.08 mg KOH/g |
| Appearance | Cloudy | Cloudy |
| Mw/Mn | 1.04 | 1.03 |
| Primary OH/Secondary OH | 50/50 | 51/49 |
| by ¹³C NMR | | |
| PO units measured by ¹³C | 6.9 | 6.7 |
| NMR | | |
| EO units measured by ¹³C | 1.7 | 1.6 |
| NMR | | |
| Co-content* | 1.7 mg/kg | 1.8 mg/kg |
| Zn-content* | 4.0 mg/kg | 4.2 mg/kg |

*measured by Inductively Coupled Plasma - Mass Spectroscopy (ICP-MS).

### Example 4

### Preparation of a Tri-Block NEODOL 67 Alcohol-Alkoxylate, Having an Average of about 7 Propyleneoxy, 2 Ethyleneoxy and 1.4 5-Hexene-2-Oxy Units Per Molecule

To a magnetically stirred 100-ml round-bottomed flask equipped with a reflux condenser, containing propoxylated-ethoxylated branched C16-17 alcohol (the product of example 2, from which the DMC catalyst had not been removed), having an average of about 7 propyleneoxy groups and 2 ethyleneoxy groups (27 g, 36 mmol), was added a DMC catalyst (intake: 200 ppm wt/wt of solid DMC catalyst based on end product) and the mixture was heated to 120° C. At this temperature 1,2-epoxy-5-hexene (5.0 g, 51 mmol) was added drop wise over 30 minutes. The resulting mixture was stirred at 120° C. for 16 hours, which upon cooling to room temperature yielded a hazy oil (32 g).

End group analysis of the thus obtained product by means of ¹H and ¹³C-NMR spectroscopy (CDCl₃, 300 MHz) showed disappearance of the ethyleneoxy end groups and appearance of signals with a chemical shift characteristic of terminal alkene groups.

### Example 5

### Preparation of a Tri-Block NEODOL 67 Alcohol-Alkoxylate, Having an Average of about 7 Propyleneoxy, 2 Ethyleneoxy and 1.4 5-Hexene-2-Oxy Units Per Molecule

A magnetically stirred 100-ml Schlenk flask, containing propoxylated-ethoxylated branched C16-17 alcohol, NEODOL67-7PO-2EO (prepared analogously to example 2, except for the amount of DMC catalyst used) having an average of about 7 propyleneoxy groups and 2 ethyleneoxy groups (27 g, 36 mmol), from which the DMC catalyst (50 ppm wt/wt on NEODOL67-7PO-2EO) had not been removed, was heated to 130° C. and evacuated for 2 hours. The mixture was then placed under a nitrogen atmosphere. Evacuation and nitrogen flushing were repeated 5 times. At 120° C., 1.4 equivalents of 1,2-epoxy-5-hexene (5.0 g, 51 mmol) which had been dried over molecular sieves (3A) and purged with nitrogen for 16 hours at room temperature, were added drop wise over 2 hours. The resulting mixture was stirred at 120° C. for 18 hours.

End group analysis of a sample of the reaction mixture by ¹H and ¹³C-NMR spectroscopy (CDCl₃, 300 MHz) showed it to be a mixture of the starting materials.

Additional DMC catalyst (intake: 55 ppm wt/wt of solid DMC catalyst based on end product) was added and the mixture was stirred at 120° C. for another 72 hours. Upon cooling to room temperature a hazy oil (31 g) was obtained.

End group analysis of the thus obtained product by means of ¹H and ¹³C-NMR spectroscopy (CDCl₃, 300 MHz) showed disappearance of the ethyleneoxy end groups and appearance of the chemical shift characteristic for a terminal alkene groups.

### Example 6

### Preparation of a Tri-Block NEODOL 67 Alcohol-Alkoxylate, Having an Average of about 7 Propyleneoxy, 2 Ethyleneoxy and 1.8 3-Chloropropoxy Units Per Molecule

Following the procedure of Example 5, an propoxylated-ethoxylated branched C16-17 alcohol, NEODOL67-7PO-2EO (prepared analogously to example 2), having an average of about 7 propyleneoxy groups and 2 ethyleneoxy groups (73.5 g, 98 mmol) and containing 100 ppm wt/wt of a solid DMC catalyst on end product, was reacted with 1.8 equivalents of nitrogen-purged 1,2-epoxy-3-chloropropane (epichlorohydrin, ECH, 16.3 g, 176 mmol) at 130° C. for 64 hours. ¹H and ¹³C-NMR spectroscopy (CDCl₃, 300 MHz) showed that >95% of the ECH had reacted and indicated the formation of 3-chloro-2-hydroxypropyl end groups.

We claim:

1. A process for the preparation of an alkoxylate composition, said process comprising the steps of:
(a) introducing into a reactor system one or more compounds with one or more active hydrogen atoms, selected from the group consisting of alkanoic acids, alkanoic amides, alkanoic ethanolamides, alcohols and alkylmercaptans, and a double metal cyanide catalyst;
(b) contacting the one or more compounds with one or more active hydrogen atoms and the double metal cyanide catalyst with propylene oxide and/or butylene oxide to form a first product mixture comprising double metal cyanide catalyst and compounds formed by the addition of one of more propylene oxide and/or butylene oxide units to the one or more compounds with one or more active hydrogen atoms; and
(c) contacting the first product mixture with ethylene oxide to form a second product mixture comprising compounds formed by the addition of one or more ethylene oxide units to the compounds formed in step (b).

2. The process of claim 1 wherein, after step (c), the second product mixture contains double metal cyanide catalyst and is contacted with a functionalised epoxide to form a third product mixture comprising compounds formed by the addition of one of more functionalised epoxide units to the compounds formed in step (c).

3. The process of claim 1 wherein the one or more compounds with one or more active hydrogen atoms is an alcohol or a mixture of alcohols.

4. The process of claim 3 wherein the alcohol or mixture of alcohols has in the range of from 8 to 36 carbon atoms.

5. The process of claim 4 wherein the alcohol has in the range of from 12 to 24 carbon atoms.

6. The process of claim 1 wherein the double metal cyanide catalyst comprises zinc hexacyanocobaltate.

7. The process of claim 3 wherein the propylene oxide or butylene oxide is contacted with the alcohol in a molar ratio in a molar ratio in the range of from 2 to 20 moles of propylene oxide or butylene oxide per mole of alcohol.

8. The process of claim 7 wherein the propylene oxide or butylene oxide is contacted with the alcohol in a molar ratio in the range of from 3 to 12 moles of propylene oxide or butylene oxide per mole of alcohol.

9. The process of claim 3 wherein the ethylene oxide is contacted with the propoxylated or butoxylated alcohol in a molar ratio in the range of from 1 to 9 moles of ethylene oxide per mole of alcohol.

10. The process of claim 3 wherein the functionalised epoxide is contacted with the ethoxylated and propoxylated or butoxylated alcohol in a molar ratio in the range of from 1 to 4 moles of functionalised epoxide per mole of alcohol.

11. The process of claim 10 wherein the functionalised epoxide is selected from the group consisting of epihalohydrins, glycidol derivatives, epoxidised acrylic or methacrylic acid derivatives and diene monoepoxides.

12. An alkoxylate composition which comprises an alcohol having been reacted with one or more molar equivalents of PO and then one or more molar equivalents of EO.

13. An alkoxylate composition which comprises an alcohol having been reacted with one or more molar equivalents of PO, then one or more molar equivalents of EO and then one or more molar equivalents of epichlorohydrin.

14. The alkoxylate composition of claim 12 wherein the alcohol is a branched primary alcohol composition having from 8 to 6 carbon atoms and an average number of branches per molecule of at least 0.7, said branching comprising methyl and ethyl branches.

15. The alkoxylate composition of claim 13 wherein the alcohol is a branched primary alcohol composition having from 8 to 6 carbon atoms and an average number of branches per molecule of at least 0.7, said branching comprising methyl and ethyl branches.

16. The alkoxylate composition of claim 14 wherein the alcohol is a branched primary alcohol composition having from 14 to 21 carbon atoms and an average number of branches per molecule of from 0.7 to 3.0, said branching comprising methyl and 5-30% ethyl branches and 5-25% branching at the carbon atom adjacent to the hydroxyl carbon atom, said composition comprising less than 0.5 atom % of quaternary carbon atoms.

17. The alkoxylate composition of claim 15 wherein the alcohol is a branched primary alcohol composition having from 14 to 21 carbon atoms and an average number of branches per molecule of from 0.7 to 3.0, said branching comprising methyl and 5-30% ethyl branches and 5-25% branching at the carbon atom adjacent to the hydroxyl carbon atom, said composition comprising less than 0.5 atom % of quaternary carbon atoms.
